(19) United States
(12) Patent Application Publication
(10) Pub. No.: US 2004/0220588 A1
(43) Pub. Date: Nov. 4, 2004

(54) Guide assembly

(76) Inventors: James Kermode, Los Altos, CA (US); Douglas Murphy-Chutorian, Palo Alto, CA (US)

Correspondence Address: OLSON & HIERL, LTD., 36th Floor, 20 North Wacker Drive, Chicago, IL, 60606, US

(21) Appl. No.: 10/427,881

(22) Filed: May 1, 2003

(51) Int. Cl. A61B 19/00

(52) U.S. Cl. 606/129

(57) ABSTRACT

A guide assembly for directing a medical instrument to a desired location in a patient is provided. The guide assembly comprises an arcuate guide support having a constant radius of curvature. The guide assembly further includes an introducer that has a hollow elongated sheath and a probe that is spaced from the introducer along the guide support. The longitudinal axes of the introducer and the probe intersect at a target location within the patient.

### TECHNICAL FIELD OF THE INVENTION

The invention relates to a guide assembly for directing a medical instrument such as a biopsy needle to a desired location in a patient.

### BACKGROUND OF THE INVENTION

Breast cancer is one of the health threats most feared by women, and is the most common form of cancer in women. A key to treatment is early detection. For example, an annual mammogram is a method that has been used in hopes of early detection of breast cancer. One problem with mammography is that such an imaging technique can only find breast cancer once it has taken form. All too often, breast cancer is discovered at a stage that is too far advanced, when therapeutic options and survival rates are severely limited. While breast cancer is most common among women, in rare instances the human male may also have occurrences of breast cancer.

Other methods of detecting breast cancer are based on the fact that in a vast majority of instances breast cancer begins in the lining of mammary ducts. Studies have shown that fluid within the mammary duct contains high levels of breast cancer markers, and that an estimated 80%-90% of all breast cancers occur within the intraductal epithelium of the mammary glands. Fluid within the breast ducts contains an assemblage and concentration of hormones, growth factors and other potential markers comparable to those secreted by, or acting upon, the surrounding cells of the alveolar-ductal system. Likewise, mammary fluid typically contains cells and cellular debris or products that can also be used in cytological or immunological assays.

Once suspicious tissue is located, procedures for eliminating or sampling the tissue are performed. For example, a biopsy of the tissue may then be taken. Cytological and histological studies of the biopsied tissue sample can then be performed as an aid to the diagnosis and treatment of disease. During the biopsy procedure, care is taken to minimize the physical trauma inflicted upon the intervening tissues that surround the affected area or target tissue and at the same time to protect the practitioner from health hazards. One typical biopsy procedure includes inserting a hollow biopsy needle through the intervening tissue into the target tissue to be sampled. One device that has been utilized for such a procedure is the Mammotome® biopsy system available from Ethicon Endo-surgery, Inc., Cincinnati, Ohio.

The first step in the biopsy procedure utilizing the Mammotome® biopsy system includes mapping the area to be biopsied through either ultrasonic imaging or stereotactic procedures. With ultrasonic imaging, a doctor obtains images of the breast tissue while the patient lies supine. In stereotactic procedures, patients lie prone on a special table. The woman's breast protrudes through an opening in the table's surface, where it is lightly compressed and immobilized while a computer produces detailed images of any abnormality present.

After a mapping an area to be biopsied, the Mammotome® probe, a needle-like device with a hollow passage therethrough, is inserted through an incision cut into the breast and inserted through the patient with a sharpened distal end until the desired biopsy area is accessed. When the probe is positioned at the area of concern, tissue is received into a window in the probe with vacuum assist. A cutter then severs and removes tissue samples for examination. The samples are passed through the hollow chamber of the probe into a collection chamber. The Mammotome® probe is removed after the samples have been collected, and the incision is closed.

Such a system decreases the invasiveness of the biopsy procedure by only requiring a small incision and puncture, which may be done under local anesthetic. However, the pre-surgical mapping procedures for guiding the insertion of the biopsy needle are complicated and expensive.

Other methods for guiding a biopsy needle have included mounting a guide upon an ultrasound transducer so that, upon insertion of the tip of the needle into a patient, the tip enters a plane that is being imaged by the transducer. The guiding of the needle is then done visually. Examples of such devices are disclosed in U.S. Pat. No. 6,527,731 issued to Weiss et al. and U.S. Pat. No. 6,475,152 issued to Kelly, Jr. et al. Again, however, such ultrasound procedures require expensive and complicated equipment to guide the biopsy needle to the desired location in a patient.

What is needed is a guide assembly for locating and guiding a medical instrument to a desired location in a patient that does not require expensive and complicated viewing or mapping equipment. The present invention meets the foregoing desires and provides an improved device for guiding medical devices to a desired location within a patient.

### SUMMARY OF THE INVENTION

A guide assembly for directing a medical instrument to a target location in a patient is provided. The guide assembly comprises an arcuate guide support having a constant radius of curvature. Preferably, the radius of curvature is about 10 centimeters (cm). The guide assembly further includes an introducer that comprises a hollow elongated sheath, and that is removably secured to the guide support. The longitudinal axis of the introducer sheath extends along the radius of curvature for the arcuate guide support. The introducer sheath is sized for insertion into a lumen, such as a mammary duct. The guide assembly further includes a probe that is also adjustably secured to the guide support spaced from the introducer. The probe comprises an elongated shaft preferably having a pointed or sharpened distal end, and having a longitudinal axis that extends along the radius of curvature of the arcuate guide support. The shaft of the probe may be hollow if desired.

The probe can also serve as an internal or external guide for a wide variety of biopsy devices, and is spaced from the introducer along the guide support as stated hereinabove. Preferably the spacing between the introducer and the probe is less than about 90 degrees of an arc, and more preferably about 10 degrees to about 30 degrees of an arc. The spacing between the introducer and the probe is preferably adjustable. The respective longitudinal axes of the introducer and the probe are also substantially coplanar with one another, such that the longitudinal axes intersect one another at about the target location. Both the introducer sheath and the probe can include spaced depth markers along the length thereof, if desired.

Preferably, the guide assembly also is suitable for use with an endoscope. The inside diameter of the introducer sheath is such that an endoscope can be readily inserted into and removed therefrom. The endoscope is extendable to about the distal end portion of the introducer sheath such that an operator may view a target location within the patient for biopsy or other medical procedure, such as ablation, marking, or the like, through an open distal end in the introducer sheath.

The endoscope may be any known microendoscope used in surgical procedures that can be accommodated by the introducer sheath. Such an endoscope can include features such as a fiber optic illuminator and a passage for introduction or removal of an irrigation liquid. In order to enhance the viewing capabilities of the endoscope, the interior of the distal end portion of the introducer sheath is preferably substantially non-reflective. The non-reflective interior can be provided by coating the interior wall of the introducer sheath, such as by anodization, blackening, or oxidation processes. Alternatively, the entire distal end portion can be formed of a non-reflective material. The substantially non-reflective interior of the distal end portion enables the illuminator of an endoscope to be positioned within the distal end portion while avoiding or at least minimizing undesirable reflective glare off the sheath or off the lumen itself, thereby enhancing the operator's view of the selected location in the lumen.

Because the guide support has a constant radius of curvature and the introducer and probe are aligned substantially along radially extending paths of the guide support, the distal end portions of the introducer and probe are at substantially the same location when the introducer and the probe are inserted into the patient at the same depth. For example, after the introducer is inserted into a mammary duct, an endoscope is extended within the introducer, and a desired location, such as a biopsy site, is located. The probe is then inserted into the patient so that the probe and introducer substantially meet at the biopsy site. Either or both the introducer and probe may be inadvertently deflected somewhat by the patient's breast tissue, therefore, the introducer and the probe have a sufficient degree of rigidity such that any such deflection is minimized and the region of access by the probe and the introducer is an within acceptable tolerances. This region of access is dependent upon the size of the tissue to be treated or biopsied.

### BRIEF DESCRIPTION OF THE DRAWINGS

In the drawings,

FIG. 1 is a schematic perspective view of a biopsy needle guide assembly embodying the present invention situated in a breast shown in cross section;

FIG. 2 is an enlarged schematic partial view of the guide assembly of FIG. 1;

FIG. 3 is an enlarged schematic partial view of the distal end portions of the introducer sheath and the probe at a target location within a patient's breast;

FIG. 3A is an enlarged schematic partial view of the distal end portions of the introducer sheath and the probe penetrating a mammary duct;

FIG. 4 is a schematic perspective view of a needle guide assembly of the present invention in a breast, which is shown in cross section, and with an illuminator carried by the probe;

FIG. 5 is an alternative embodiment of the present invention, showing a needle guide assembly equipped with a biopsy needle mounted to the arcuate guide support; and

FIG. 6 is an enlarged partial perspective view of an introducer sheath, partially broken away to show interior detail having an endoscope positioned therewithin, and the interior wall of the sheath provided with a non-reflective coating.

### DESCRIPTION OF THE PREFERRED EMBODIMENT OF THE INVENTION

The invention disclosed herein is susceptible of embodiment in many different forms. Shown in the drawings and described hereinbelow in detail are preferred embodiments of the invention. It is to be understood, however, that the present disclosure is an exemplification of the principles of the invention and does not limit the invention to the illustrated embodiments.

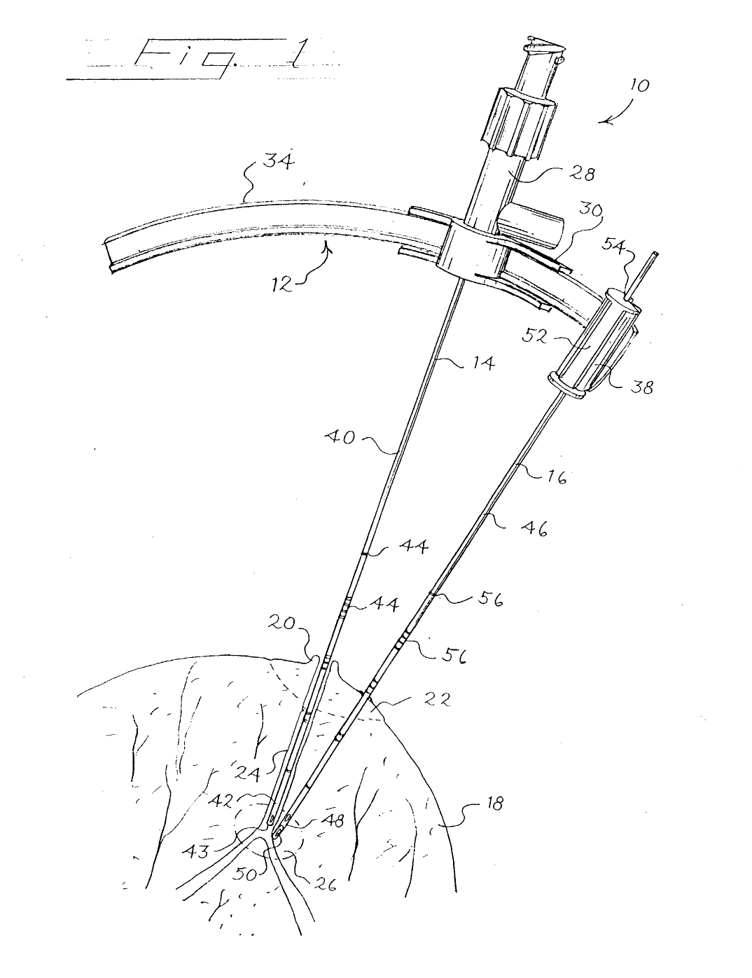

A preferred embodiment of the guide assembly 10 is shown in FIG. 1. Guide assembly 10 comprises an arcuate guide support 12 having a constant radius of curvature. In a preferred embodiment, the constant radius of curvature is approximately 10 cm. The guide assembly 10 further includes an introducer 14 slidably mounted to guide support 12 and a probe 16 also mounted to guide support 12. As shown the introducer 14 is inserted into a human breast 18. For example, the introducer 14 is inserted into a mammary duct 24 by way of a mammary duct orifice at the nipple 20. The probe 16 is also inserted into breast 18 and breast tissue, but through a small incision made in the breast 18. Preferably, the incision is made in the areola 22 to minimize visible scarring. The probe 16 is spaced from the introducer 14 along the guide support 12. The spacing between the introducer and the probe is adjustable, preferably less than about 90 degrees of an arc, and more preferably in the range of about 10 degrees to about 30 degrees of an arc. The introducer 14 can be held in place on guide support 12 by friction or using a releasable clip.

The introducer 14 and the probe 16 are also substantially coplanar with one another. The longitudinal axis of introducer 14 substantially intersects with the longitudinal axis probe 16 at the desired target location 26 as shown. In other words, the respective longitudinal axis of the introducer 14 and the probe 16 are substantially along radial paths of the circle defined in part by the arcuate guide support 12.

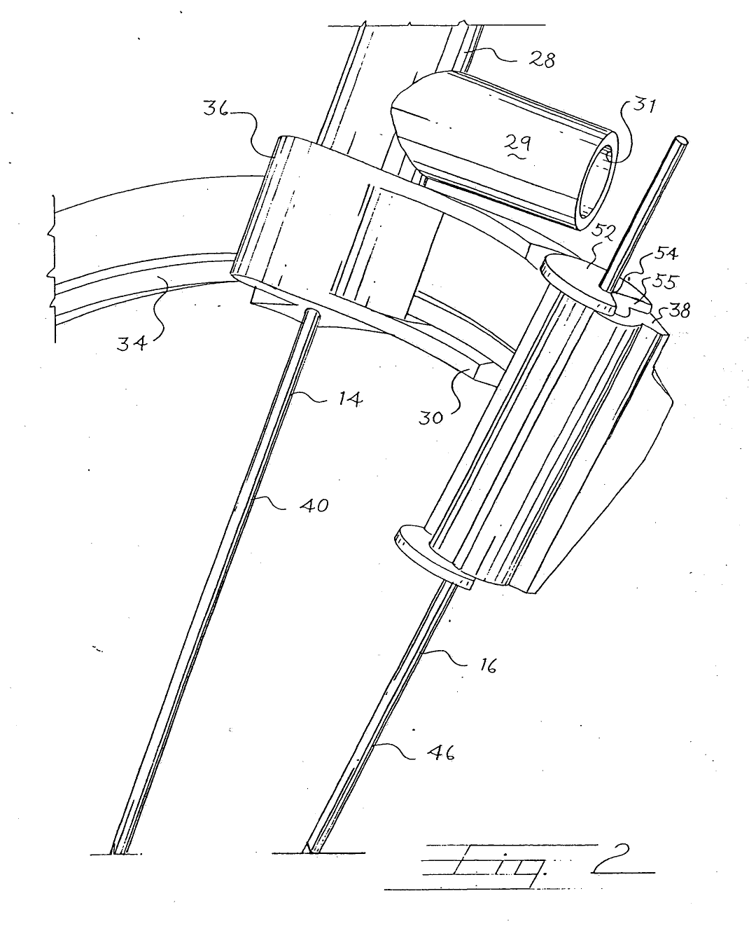

Referring to FIGS. 1 and 2, the guide support 12 comprises a carriage 30, which is in sliding engagement with arcuate track 34. A holster 36 for removably securing introducer 14 to the guide assembly 10 is carried by guide support 12. Preferably, the holster 36 is integral with carriage 30. In this particular embodiment, the holster 36 cooperates with track 34 to hold the introducer 14 in place. A bracket 38 for removably securing the probe 16 to guide support 12 is provided at one end of track 34. The bracket 38 may take on various configurations, as desired. For example, the bracket 38 may include a through passage for guiding probe 16. It is preferred however, that the probe 16 be in sliding engagement with a releasable collar 52 having a passage 54 defined therewithin through which probe 16 is passed. In this way, probes of different outside diameters can be accommodated by a particular guide support through use of multiple collars or a single collar adapted for accepting probes of various diameters that are removably securable to and orientable with the bracket 38. Access to passage 54 may be facilitated by notch 55. Preferably, the guide support is made of polypropylene, polyethylene, nylon, or other suitable plastic material of construction. Holster 36 can also accommodate microendoscope positioning hub 28 equipped with side stem 29 that is hollow and terminates in irrigation port 31. Probe 16 can also be any existing type of pointed biopsy or interventional therapeutic tool, such as the Mammotome™ biopsy device discussed above. Alternatively, a pointed probe 16 can be introduced through bracket 38 and serve as an internal or external guide pathway for other biopsy or therapeutic tools.

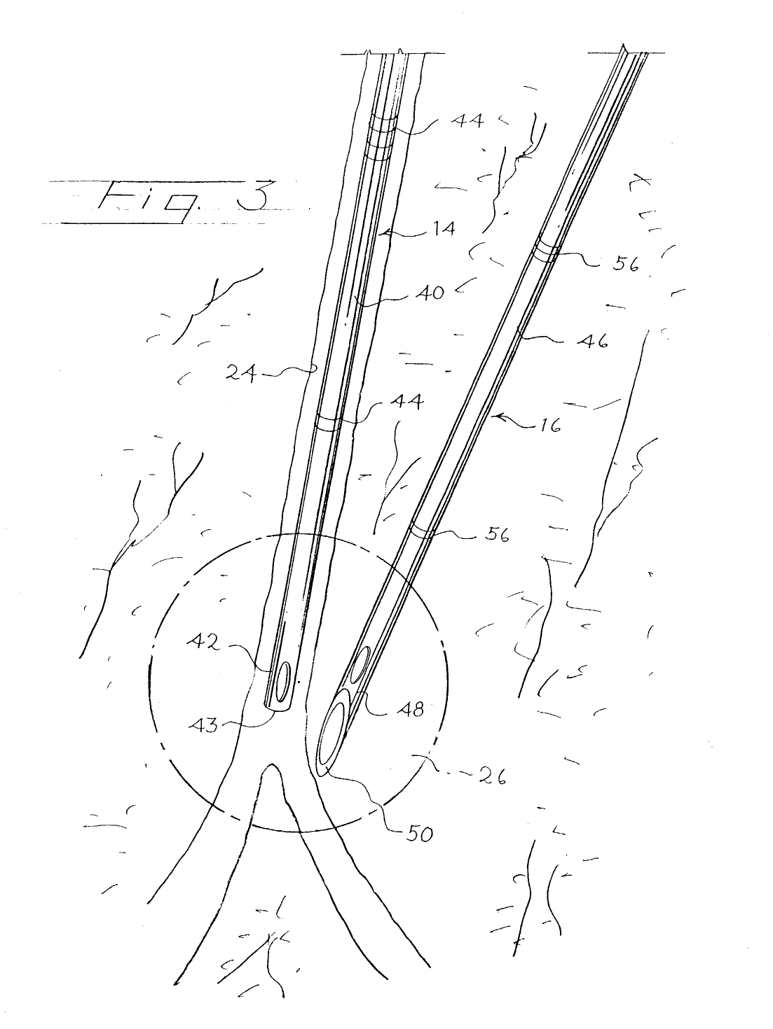

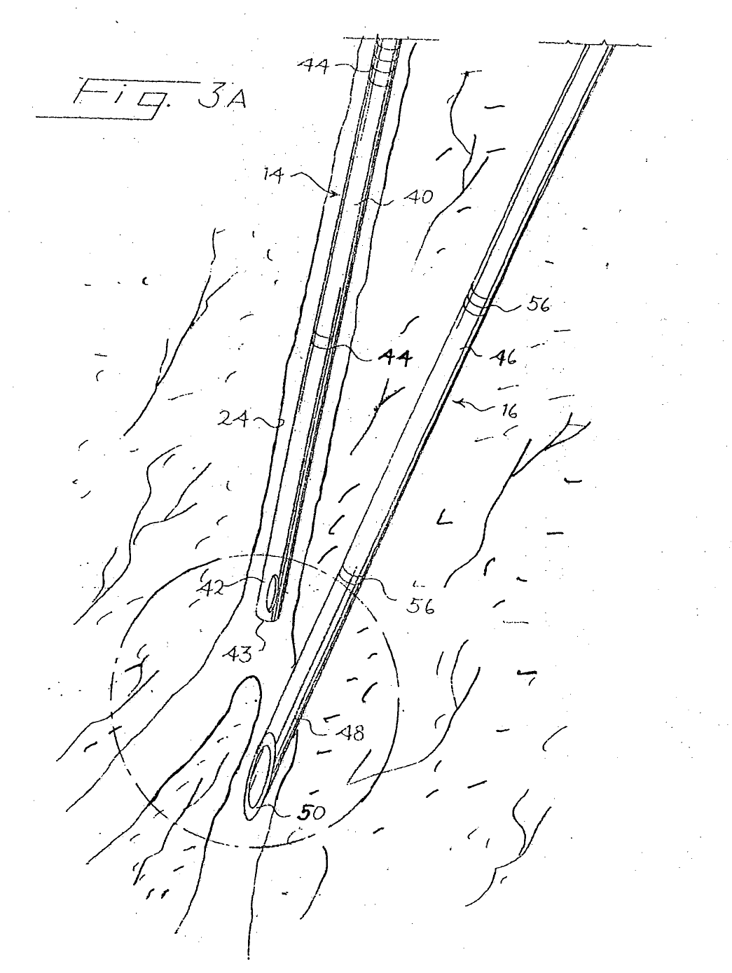

Referring to FIG. 3, the introducer 14 comprises a hollow elongated sheath 40. The longitudinal axis of sheath 40 substantially follows a radial path of the circle partially defined by the arcuate guide support 12. Depth markers 44 are included on the sheath 40 to indicate to the operator the extent to which the introducer 14 has been inserted into a body lumen such as mammary duct 24. Introducer 14 further includes a distal end portion 42 and an open distal end 43. The distal end portion 42 may also include a side cut-out or aperture to permit tissue to be excised within the aperture so as to be cut with a biopsy cutter.

Probe 16 is comprised of an elongated shaft 46, which can either be hollow or solid depending on whether the instrument being guided to the target site is to be extended through the shaft 46 or introduced over the shaft 46. Alternatively, the probe itself may be a diagnostic or therapeutic device, such as the Mammotome™ device discussed above. As shown, the shaft 46 is hollow and further includes a distal end portion 48 having a sharpened end 50. An example of a solid probe is a guide wire or rod (not shown) over which a desired instrument can be introduced at a predetermined site in the tissue. Optional depth markers 56 are provided on shaft 46. Examples of diagnostic and therapeutic devices that may be used as or guided by the probe 16 include ablation devices, biopsy devices, marking devices, cutters, nibblers, and suction devices. Similar to the introducer 14, the distal end portion 48 may also include a side cut-out or aperture to permit tissue to be excised within the aperture so as to cut with a biopsy cutter.

An additional method of using guide assembly 10 is shown in FIG. 3A. The distal end 48 of probe 16 can be positioned within mammary duct 24 by penetrating the mammary duct. In this particular example, the distal end 48 is below the distal end 43 of the introducer 14 and serve as a fluid return path for downstream irrigation fluid or tissue recovery by suction. For example, an intraductal shaver or brush can be provided through introducer 14 to brush samples of the epithelium. The collection of the brushed samples, along with any irrigation fluid also provided through the introducer, can be collected via probe 16, such as by a vacuum source operatively connected thereto.

The guide assembly 10 is suitable for use with an endoscope positioning hub 28. An endoscope mounted to hub 28 can be extended to about the distal end portion 42 of the introducer 14 sheath 40 so that an operator may view a desired location 26 within the patient for biopsy or other medical procedure, such as ablation, marking, or the like, through an open distal end 43 in the introducer sheath 40.

The endoscope may be any known microendoscope used in surgical procedures that can be accommodated by the introducer sheath 40. Such an endoscope can include features such as a fiber optic illuminator, a passage for introduction or removal of liquid. A working end effector can also be included with the endoscope such as a nibbler or cutter.

In use each of the probe 16 and the introducer 14 are extended along their respective longitudinal axis and substantially along radial paths of a circle defined in part by arcuate guide support 12. Introducer 14 is extended first to select the target site. Thereafter probe 16 is introduced into the tissue. Introducer 14 and probe 16 meet at the target location 26 in the patient, which approximates the center of the circle. Either or both the introducer or probe may be inadvertently deflected somewhat by the patient's breast tissue; therefore, introducer and probe should have adequate rigidity to minimize such deflection.

The introducer 14 can be inserted into a body lumen such as mammary duct 24 via a nipple orifice. The nipple orifice may be located by any convenient means, such as an illuminated nipple cup (not shown), or the like. A conventional nipple orifice dilator or catheter may be used to dilate the nipple orifice, if desired, to facilitate insertion of the introducer 14. An endoscope is preferably inserted within the introducer 14 for inspection of the target location 26. After the target location 26 is located within the mammary duct, the probe 16 is inserted into the patient through an incision, and is guided by the collar 52 and bracket 38 secured to the guide support 12. As discussed, the constant radius of curvature of the guide support 12 directs the probe 16 and the introducer 14 along radial paths of the same circle toward the same location. In this manner, the tips of probe 16 and introducer 14 meet at substantially the same location. Preferably, the accuracy of the guide assembly 10 is such that the introducer 14 and probe 16 converge within a region of about 1 cubic centimeter, and more preferably to within a region of about 0.5 cubic centimeter. The depth to which the probe 16 is inserted can be approximated by depth markers 56, which correlate with depth markers 44 on the introducer 14.

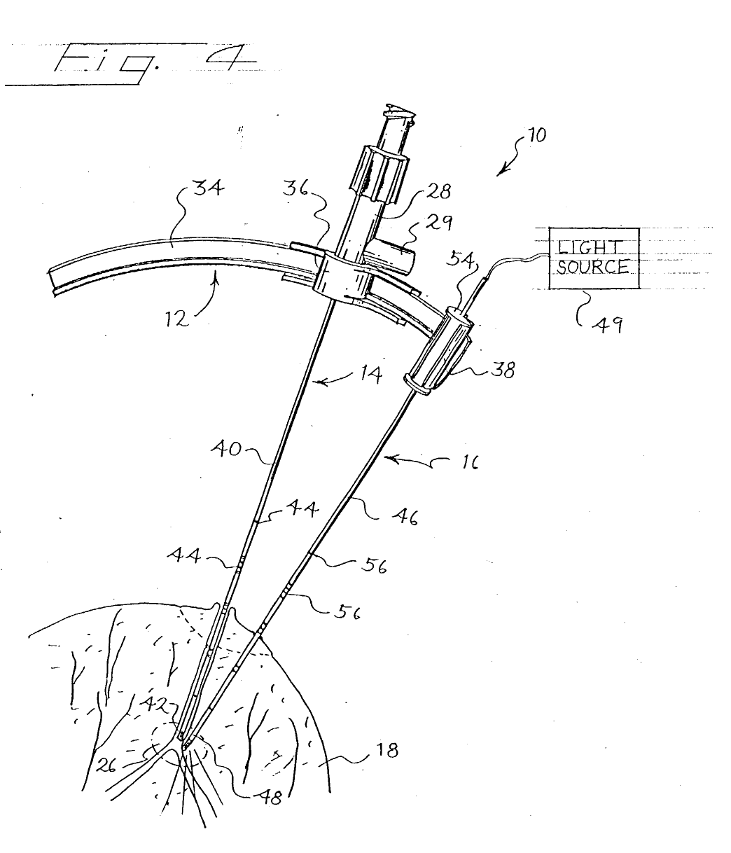

Probe 16 is also suitable for introducing an independent fiber optic illuminator into the breast as shown schematically in FIG. 4. The illuminator within probe 16 is coupled to a light source 49 and can be extended to the distal end portion 48 of the probe 16 such that light illuminates a region about the distal end of the probe 16. As the probe 16 is guided toward the target location 26, where the distal end portion 42 of the introducer 14 is already situated, the light emanating from the distal end portion of probe 16 will be partially visible through the breast tissue by an endoscope in introducer 14. The breast 18 can then be manipulated by the operator to more precisely guide the probe 16 to the target location 26. Alternatively, an illuminator can be integral with the probe. Other ways of sensing the probe include electrical resistance sensors, ultrasound mechanisms, and the like. The particular detector used can be extended through the introducer to the distal end portion thereof, or can alternatively be provided as part of the introducer itself It should also be noted that the sensor can be provided by either the introducer or probe. By way of illustration, in the previous example with the fiber optic illuminator and the endoscope, the endoscope can alternatively be extended within the probe and the illuminator extended within the introducer.

After the distal end portions 42 and 48 of the introducer 14 and probe 16, respectively, are positioned, the introducer can serve as a conduit for any number of additional devices complementary to the devices guided by the probe 16. For example, the introducer 14 may act as a flush lumen or as a guide for a nibbler or cutter.

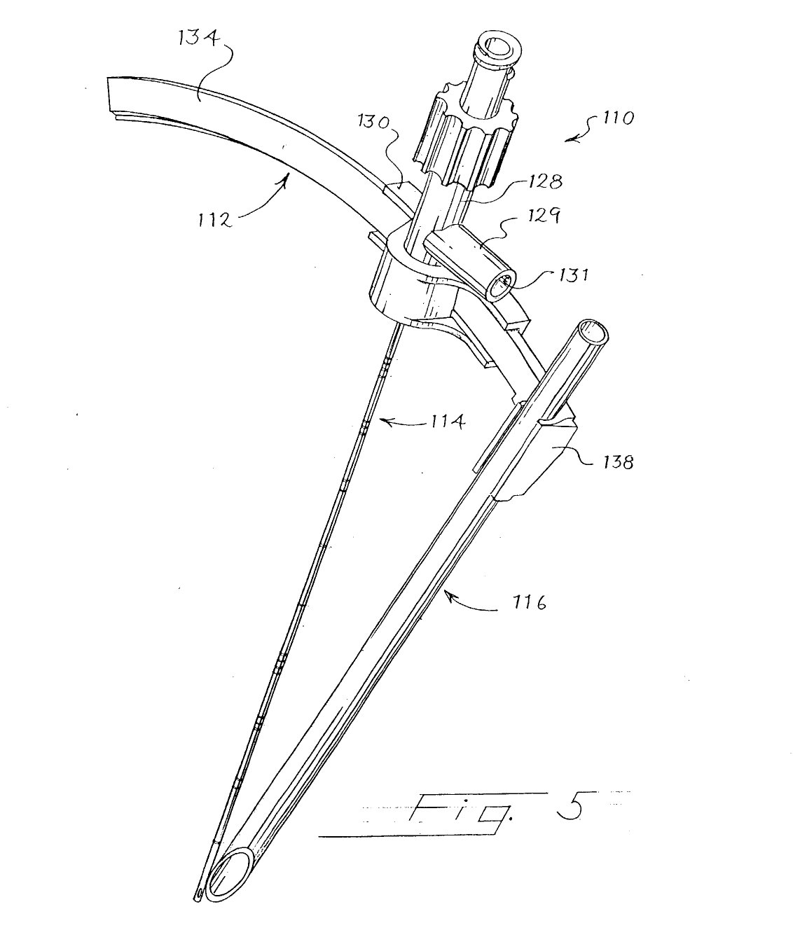

An alternative embodiment of a guide assembly is shown in FIG. 5. Generally, this embodiment is quite similar to the previous embodiment. Guide assembly 110 comprises an arcuate guide support 112 having a constant radius of curvature, an introducer 114 and a probe 116. Introducer 114 is received in holster 136 which is part of carriage 130 slidably mounted to track 134. Hub 128 from mounting and positioning a microendoscope is provided at the proximal end of introducer 114. Stem 129 is in fluid communication with hub 128 and can receive an irrigation fluid via access port 131. At one end of the rail 134 is a bracket 138 for removably securing the probe 116 to guide support 112. In this embodiment, unlike that shown in FIGS. 1 and 2, no collar is utilized. Instead, the bracket 138 directly pinches the probe 116. This embodiment illustrates the wide range of probes that may be utilized depending on the instrument to be guided.

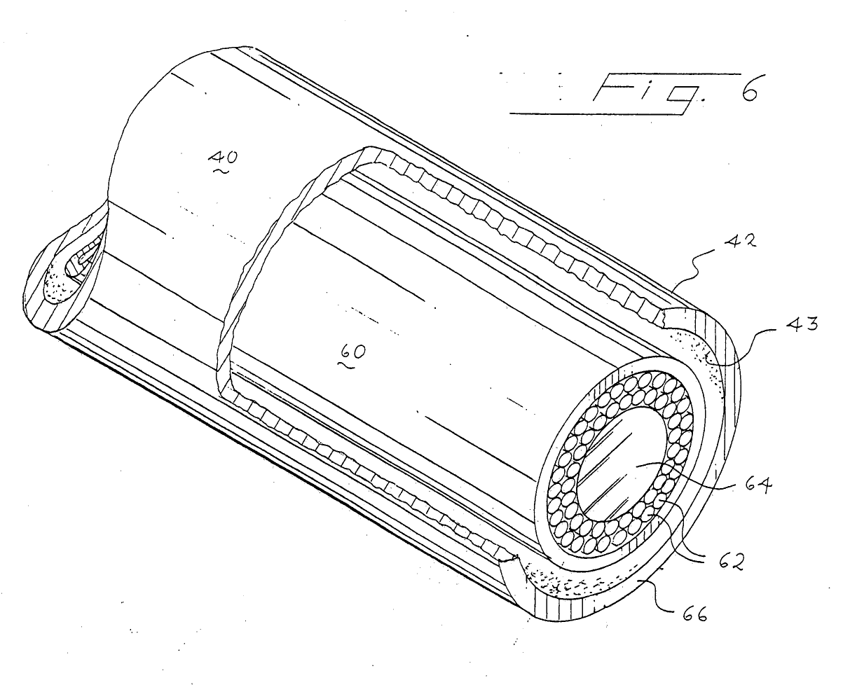

In order to enhance the viewing capabilities of the endoscope, the distal end portion 42 of the introducer sheath 40 preferably includes an interior portion 43 that is substantially non-reflective, as shown in FIG. 6. This may be done by coating at least the distal end portion 42 of the sheath interior, such as by anodization, blackening, or oxidation processes. Alternatively, the distal end portion 42 of introducer sheath 40 can be formed of a non-reflective material. This substantially non-reflective distal end portion 43 enables the illuminator 62 of an endoscope 60 to be positioned within the distal end portion such that reflective glare off the interior of the sheath 40, or off the lumen, such as mammary duct 24, is eliminated or greatly reduced, thereby enhancing the operator's viewing of the desired location in the lumen through endoscope lens 64. The non-reflective interior portion 43 of introducer sheath 42 preferably is at least about one introducer sheath inside diameter long as measured from the distal end 66 of sheath 42. That is, for an introducer sheath having an inside diameter of about 0.032 inches (about 0.8 millimeters) the non-reflective interior portion extends inwardly from the introducer sheath distal end about 0.032 inches (about 0.8 millimeters), more preferably about 0.05 inches (about 1.25 millimeters). A black oxide coating is particularly well suited for providing a non-reflective coating on interior portion 43. Compositions for stainless steel that can be used for this purpose are PX-5 commercially available from HeatBath Corporation, Indian Orchard, Mass., Insta-Blak SS-370 commercially available from Electrochemical Products, Inc. New Berlin, Wis., and the like.

The foregoing descriptions are to be taken as illustrative, but not limiting. Still other variants within the spirit and scope of the present invention will readily present themselves to those skilled in the art.

What is claimed is:

1. A guide assembly for directing a medical instrument to a target location in a patient, the guide assembly comprising:
an arcuate guide support having a constant radius of curvature;
an introducer removably secured to the guide support, the introducer comprising a hollow elongated sheath terminating in a distal end portion suitable for insertion into a body lumen;
a probe spaced from the introducer removably secured to the guide support, and comprising an elongated shaft having a sharpened distal end portion;
the longitudinal axes of the introducer and the probe being coplanar and intersecting at the target location.

2. The guide assembly of claim 1, wherein the introducer further comprises spaced depth markers on the elongated sheath.

3. The guide assembly of claim 1, wherein the probe further comprises spaced depth markers along the shaft thereof.

4. The guide assembly of claim 1, wherein the introducer has an open distal end.

5. The guide assembly of claim 1, wherein the introducer distal end portion has a substantially non-reflective interior.

6. The guide assembly of claim 5, wherein the substantially non-reflective interior includes a non-reflective coating.

7. The guide assembly of claim 6, wherein the substantially non-reflective coating is an anodized coating.

8. The guide assembly of claim 6, wherein the substantially non-reflective coating is an oxide.

9. The guide assembly of claim 5, wherein the non-reflective interior extends inwardly from the distal end of the introducer sheath for a length equal to at least one inside diameter of the introducer sheath.

10. The guide assembly of claim 1, wherein the distal end portion is comprised of a substantially non-reflective material.

11. The guide assembly of claim 1, wherein the probe is removably secured to the guide support by a bracket.

12. The guide assembly of claim 11, wherein the probe further includes a collar defining a through passage for the sheath, the collar being removably secured to the bracket.

13. The guide assembly of claim 1, wherein the probe is adjustably spaced from the introducer along the arcuate guide support less than about 90 degrees of an arc.

14. The guide assembly of claim 13, wherein the probe is adjustably spaced from the introducer along the guide support about 10 degrees to about 30 degrees of an arc.

15. The guide assembly of claim 1, wherein the radius of curvature is about 10 centimeters.

16. The guide assembly of claim 1, wherein the probe is hollow.

17. The guide assembly of claim 16, wherein the probe has an open distal end.

18. The guide assembly of claim 16, wherein the probe distal end portion further includes a side aperture.

19. The guide assembly of claim 16, wherein the probe is a diagnostic device.

20. The guide assembly of claim 16, wherein the probe is a therapeutic device.

21. The guide assembly of claim 16, wherein the probe is suitable for introducing a liquid into a mammary duct.

22. The biopsy device of claim 16, wherein the probe is suitable for removing a liquid from the mammary duct.

23. The guide assembly of claim 1, wherein a detector is provided about the distal end portion of the introducer, and wherein the detector senses a location of the distal end portion of the probe.

24. The guide assembly of claim 23, wherein the detector is an endoscope.

25. The guide assembly of claim 24, wherein an illuminator is extended to the distal end portion of the probe.

26. The guide assembly of claim 24, wherein an illuminator is provided about the distal end portion of the probe.

27. The guide assembly of claim 23, wherein the detector is an electrical resistance sensor.

28. The guide assembly of claim 23, wherein the detector is an ultrasound device.

29. The guide assembly of claim 1, wherein a detector is provided about the distal end portion of the probe, and wherein the detector senses a location of the distal end portion of the introducer.

30. The guide assembly of claim 29, wherein the detector is an endoscope.

31. The guide assembly of claim 30, wherein an illuminator is extended to the distal end portion of the introducer.

32. The guide assembly of claim 30, wherein an illuminator is provided about the distal end portion of the introducer.

33. The guide assembly of claim 29, wherein the detector is an electrical resistance sensor.

34. The guide assembly of claim 29, wherein the detector is an ultrasound device.

35. The guide assembly of claim 1, wherein the introducer further includes a sharpened distal end for piercing tissue.

36. The guide assembly of claim 1, wherein the probe is a guide wire.

37. The guide assembly of claim 36, wherein a diagnostic or therapeutic device is guided over the guide wire.

38. The guide assembly of claim 1, wherein the probe is a guide rod.

39. The guide assembly of claim 38, wherein a diagnostic or therapeutic device is guided over the guide rod.

40. The guide assembly of claim 1, wherein the target location is about 1 cubic centimeter.

41. The guide assembly of claim 1, wherein the target location is less than 1 cubic centimeter.

42. The guide assembly of claim 1, wherein the target location is about 0.5 cubic centimeters to about 1 cubic centimeter.

43. An introducer suitable for receiving an endoscope having an illuminator, the introducer comprising:
an elongated hollow sheath sized for insertion into a body lumen and having a distal end portion terminating in an open end; and
the distal end portion having a substantially non-reflective interior.

44. The introducer of claim 43, wherein the interior of the distal end portion has a non-reflective coating.

45. The introducer of claim 44, wherein the coating is an anodized coating.

46. The introducer of claim 44, wherein the coating is an oxide coating.

47. The introducer of claim 43, wherein the distal end portion is made of a non-reflective material.

48. The introducer of claim 44, wherein the sheath is substantially rigid.

49. The introducer of claim 44, wherein the substantially non-reflective interior extends inwardly along the sheath from the distal end thereof for a distance equal to at least one inside diameter of the sheath.

50. A method for directing a medical instrument to a target location within a lumen of a patient using a guide assembly of claim 1, the method comprising the steps:
extending an introducer along a first longitudinal axis to the target location; and
extending a probe along a second longitudinal axis to the target location.

51. The method of claim 50 wherein a detector is provided about the distal end portion of the introducer, the method including the further step of detecting location of the distal end portion of the probe.

52. The method of claim 50, further including the step of guiding a diagnostic or therapeutic device to the target location with the probe.

53. The method of claim 52, wherein the probe is hollow and the diagnostic or therapeutic device is guided to the target location by extending the diagnostic or therapeutic device through the probe.

54. The method of claim 52, wherein the probe is a guide wire and the diagnostic or therapeutic device is guided to the target location by extending the diagnostic or therapeutic device along the probe.

55. The method of claim 52, wherein the probe is a guide rod and the diagnostic or therapeutic device is guided to the target location by extending the diagnostic or therapeutic device along the probe.

56. The method of claim 50, further including the step of extending a distal end portion of the probe into the lumen.

57. The method of claim 50, wherein the probe is extended through a wall of the lumen.

58. The method of claim 50, wherein the probe is suitable for removing fluid from the lumen.

59. The method of claim 50, wherein the probe is suitable for introduction of an irrigation fluid to the lumen.

60. The method of claim 50, further including the step of conducting a diagnostic or therapeutic procedure with the probe.

61. The method of claim 50, further including the step of making an incision in the patient and extending the probe into the patient through the incision.
